(19) United States
(12) Patent Application Publication
(10) Pub. No.: US 2005/0222461 A1
(43) Pub. Date: Oct. 6, 2005

(54) Method for the production of substituted trifluoroethylenes

(76) Inventors: Peter Wolfrum, Monheim (DE); Albrecht Marhold, Leverkusen (DE)

Correspondence Address: BAYER CROPSCIENCE LP;Patent Department, 100 BAYER ROAD, PITTSBURGH, PA, 15205-9741, US

(21) Appl. No.: 10/514,345

(22) PCT Filed: May 8, 2003
(86) PCT No.: PCT/EP03/04809

§ 371 Date: Apr. 29, 2005

(30) Foreign Application Priority Data
May 13, 2002 (DE) 102 21 120.5

(51) Int. Cl. C07C 21/18; C07C 231/12

(52) U.S. Cl. 564/209; 570/155

(57) ABSTRACT

The present invention relates to a method for the production of substituted trifluoroethylenes, in which a compound of the formula (II) 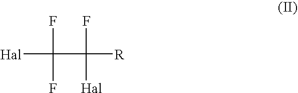 in which
R is C₁-C₆-alkyl optionally substituted by carboxyl, halogen, C₁-C₆-alkoxy, C₁-C₆-alkoxycarbonyl or C₁-C₄-alkylcarbonyloxy or is the grouping 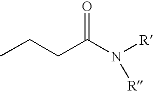
   in which R′ and R″ are, independently of one another, hydrogen or C₁-C₆-alkyl or the two, together with the nitrogen atom to which they are bonded, are saturated cycloalkyl with 3 to 5 carbon atoms, in which a ring member is optionally replaced by oxygen or sulfur and which is optionally substituted by one to four C₁-C₄-alkyl, trifluoromethyl or C₁-C₄-alkoxy, and Hal is chlorine, bromine or iodine, preferably chlorine or bromine, is brought into contact with a dehalogenating agent in a water-comprising polar medium.

The present invention relates to a method for the production of substituted trifluoroethylenes.

Trifluoroethylenes have been known for some time. Thus, trifluoroethylenes, such as, e.g., 1,1,2-trifluoro-4-bromo-1-butene, are important intermediates in the production of agrochemicals, in particular compounds having an insecticidal and nematicidal effect (cf. U.S. Pat. No. 3,513,172, WO 86/07590, EP 342 150 A1, EP 507 464 A1, WO 02/06260, WO 02/06256, WO 02/06257 and WO 02/06259).

Trifluoroethylenes can be reduced, e.g., by dehalogenation of trifluorodihaloethane derivatives. Thus, Tarrant et al. (J. Org. Chem., 34 (2), 323 (1969)) describe the dehalogenation of 1,4-dibromo-2-chloro-1,1,2-trifluorobutane using zinc in ethanol. EP 334 796 A1 discloses the dehalogenation of the same starting material electro-chemically in methanol. A common feature of both methods is that the reaction is carried out in a short-chain alcohol. However, the methods known hitherto for the production of these intermediates exhibit a number of disadvantages. Thus, the yields, e.g., are relatively low. It was therefore an object of the present invention to make available a method which makes possible the production of trifluoroethenes with good yields and with simple purification.

An improved method for the production of substituted trifluoroethylenes of the formula (I) has now been found, 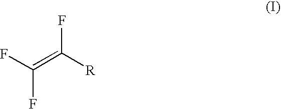
in which
R is C₁-C₆-alkyl optionally substituted by carboxyl, halogen, C₁-C₆-alkoxy, C₁-C₆-alkoxycarbonyl or C₁-C₄-alkylcarbonyloxy or is the grouping 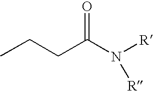
   in which
   R′ and R″ are, independently of one another, hydrogen or C₁-C₆-alkyl or the two, together with the nitrogen atom to which they are bonded, are saturated cycloalkyl with 3 to 5 carbon atoms, in which a ring member is optionally replaced by oxygen or sulfur and which is optionally substituted by one to four C₁-C₄-alkyl, trifluoromethyl or C₁-C₄-alkoxy,
      characterized in that a compound of the formula (II) 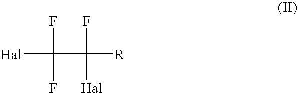
      in which
R has the abovementioned meaning, and
Hal is chlorine, bromine or iodine, preferably chlorine or bromine,
is brought into contact with a dehalogenating agent in a water-comprising polar medium.

Formula (I) also includes the metal salts, in particular of the carboxylic acids according to the invention.

Particularly preferred definitions of the compounds of the formula (I) which are produced using the method according to the invention are listed below.
R is preferably methyl, ethyl, n-propyl, isopropyl, n-butyl, isobutyl, s-butyl or t-butyl, in each case optionally substituted by carboxyl, fluorine, chlorine, bromine, methoxy, ethoxy, n-propoxy, isopropoxy, n-butoxy, isobutoxy, s-butoxy, t-butoxy, n-pentoxy, isopentoxy, methoxycarbonyl, ethoxycarbonyl, n-propoxycarbonyl, isopropoxycarbonyl, n-butoxycarbonyl, isobutoxy-carbonyl, s-butoxycarbonyl, t-butoxycarbonyl, methylcarbonyloxy, ethyl-carbonyloxy, n-propylcarbonyloxy, isopropylcarbonyloxy, n-butylcarbonyloxy, isobutylcarbonyloxy, s-butylcarbonyloxy or t-butylcarbonyloxy.
R is furthermore preferably the grouping 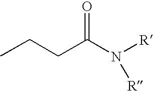
   in which
   R′ and R″ are, independently of one another, hydrogen, methyl, ethyl, n-propyl, isopropyl, n-butyl, isobutyl, s-butyl or t-butyl or the two, together with the nitrogen atom to which they are bonded, are one of the following rings 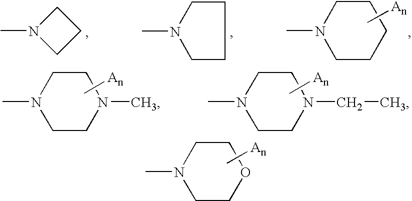
      in which
      A is methyl, ethyl, n-propyl, isopropyl or s-propyl, and
      n is 0, 1, 2, 3 or 4.
R is particularly preferably methyl or ethyl, in each case optionally substituted by carboxyl, fluorine, chlonine, bromine, methoxy, ethoxy, n-propoxy, isopropoxy, n-butoxy, isobutoxy, s-butoxy, t-butoxy, n-pentoxy, isopentoxy, methoxycarbonyl, ethoxycarbonyl, n-propoxycarbonyl, isopropoxycarbonyl, n-butoxycarbonyl, isobutoxycarbonyl, s-butoxycarbonyl, t-butoxycarbonyl, methylcarbonyloxy, ethylcarbonyloxy, n-propylcarbonyloxy, isopropyl-carbonyloxy, n-butylcarbonyloxy, isobutylcarbonyloxy, s-butylcarbonyloxy or t-butylcarbonyloxy.
R is furthermore particularly preferably the grouping 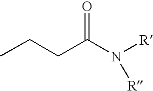
   in which
   R′ and R″ are, independently of one another, hydrogen, methyl or ethyl or the two, together with the nitrogen atom to which they are bonded, are one of the following rings

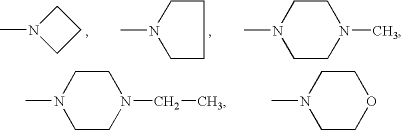

Suitable polar media (solvents) also include, in addition to water, mixtures of water with a solubility promoter. The following compounds can, e.g., be used as solubility promoters (cosolvents), the list not being definitive: ethers, such as tetrahydrofuran, diethylene glycol monomethyl ether, dioxane or bis(2-methoxyethyl) ether (diglyme), amides, such as formamide, DMF, dimethylacetamide or 1-methyl-2-pyrrolidinone (NMP), alcohols, such as methanol, ethanol, ethylene glycol, diethylene glycol, propylene glycol or glycerol, acids, such as acetic acid, propionic acid or methoxyacetic acid, ketones, such as acetone or methyl ethyl ketone, esters, such as ethyl acetate or methoxyethyl acetate, carbonates, such as diethyl carbonate, ethylene carbonate or propylene carbonate, ureas, such as N,N-dimethylethyleneurea or N,N-dimethylpropyleneurea, sulfones, such as tetramethylene sulfone, or sulfonamides, such as N,N-dimethylmethanesulfonamide or N,N-dimethyl-ethanesulfonamide.

Particularly preferred solvents which are used in the method according to the invention are water or mixtures of water with ethers, e.g. diethylene glycol monomethyl ether, or polyalcohols, e.g. diethylene glycol.

The ratio (v/v) of the water-comprising polar medium to the solubility promoter is preferably 1:10 to 1:100, particularly preferably 1:15 to 1:40.

Suitable dehalogenating agents include, e.g., metals, such as iron, zinc, magnesium, aluminum, tin, copper or nickel. Iron and/or zinc are particularly suitable for use in the method according to the invention. Mixtures of the metals or forms activated by addition of metal can also be used according to the invention.

The reaction can be carried out within a broad temperature range. It is preferably carried out at temperatures of 0 to 100° C., temperatures of 10 to 90° C. being preferred.

The method according to the invention makes it possible, in all cases where the product is readily volatile, to isolate the desired product of the formula (I) directly by distillation from the reaction solution. For example, azeotropically entrained water can be separated in the distillate simply by phase separation. The compounds of the formula (I) can subsequently be easily dried, thus, e.g., by addition of some molecular sieve or other drying agents.

The starting compounds of the formula (II) can be produced according to methods known per se, e.g. analogously to U.S. Pat. No. 3,562,341; Chin. J. Chem., 281 (1990); J. Org. Chem., 29, 1198 (1964); J. Fluorine Chem., 66, 171 (1994); Tetrahedron Lett., 31, 1307 (1990).

The following examples illustrate the method according to the invention with different starting compounds and different solvents and dehalogenating agents. The method according to the invention can also, in an analogous way, be carried out with other abovementioned starting compounds, solvents and dehalogenating agents. The examples are therefore not to be interpreted in a limiting fashion.

### EXAMPLE 1

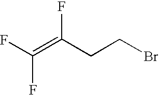 131 g (2 mol) of zinc dust are introduced into 1000 ml of diethylene glycol and 50 ml of water in an apparatus equipped with a stirrer and are suspended by stirring. After warming to 50° C., 500 g (1.64 mol) of 1,4-dibromo-2-chloro-1,1,2-trifluorobutane are metered in inside 2 hours. A slightly exothermic reaction arises and the metering rate is adjusted so that the internal temperature is maintained at 50-55° C. without additional external warming. After the reactant has finished being metered in, stirring is carried out for a further 30 min, then vacuum is applied and the product is distilled. The bulk of the product distills over at a boiling point of 46-48° C./200 mbar.

The yield of 1,1,2-trifluoro-4-bromo-1-butene is 312 g, content 90%, which corresponds to a yield of 90% of theory.

### EXAMPLE 2

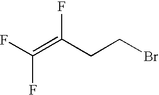 104 g (1.59 mol) of zinc, 1 g of ZnCl₂ and 500 ml of diethylene glycol monomethyl ether, together with 20 ml of water, are introduced into an apparatus equipped with a stirrer. 250 g (0.82 mol) of 1,4-dibromo-2-chloro-1,1,2-trifluorobutane are added dropwise at an internal temperature of 45° C. After the reaction temperature has risen to 50° C., the amount metered in is adjusted so that the reaction takes place at 50 to 52° C. After the end of the addition, the mixture is stirred for an additional 40 min and the pressure is subsequently reduced until the product distills off via a bridge-type stillhead.

The yield of 1,1,2-trifluoro-4-bromo-1-butene is 148 g and the content, by GC analysis, is 92.7%.

### EXAMPLE 3

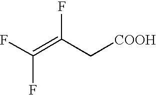 100 g of iron (powder and fine filings mixed) are introduced into 700 ml of water in an apparatus equipped with a stirrer, heating is carried out to 90° C. and, subsequently, 255 g of 4-bromo-3-chloro-3,4,4-trifluorobutanecarboxylic acid are metered in at this temperature. The temperature is maintained at approximately 90° C., the mixture is stirred for an additional 4 hours after the end of the addition, is cooled down and is brought to a pH of 1 by addition of concentrated sulfuric acid, and the product is extracted twice with 300 ml of dichloromethane each time. After distilling off the solvent, 101 g of 3,4,4-trifluoro-3-butenecarboxylic acid remain.

### EXAMPLE 4

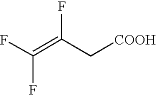

Analogously to Example 3, 12.3 g of zinc dust are introduced into 41 ml of water at 0° C. and 48.5 g of 4-bromo-3-chloro-3,4,4-trifluorobutanecarboxylic acid are added dropwise. The reaction is slightly exothermic and the temperature climbs to 10° C. After the end of the addition, a further 4.3 g of zinc dust are again added. Stirring is carried out for a further 2 hours, the pH is then adjusted to 2 with 6N hydrochloric acid and extraction is carried out twice with dichloromethane. The combined organic phases are then washed with 6N hydrochloric acid and subsequently dried over magnesium sulfate. After distilling off the dichloromethane, 19.8 g of crude product remain and comprise, according to GC analysis, 17.1 g of product (yield 63.9% of theory).

1-7. (canceled)

8. A method for the production of compounds of formula (I) 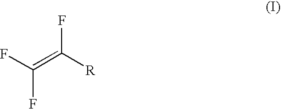
in which R is C₁-C₆-alkyl optionally substituted by carboxyl, halogen, C₁-C₆-alkoxy, C₁-C₆-alkoxycarbonyl, or C₁-C₄-alkylcarbonyloxy or is the group 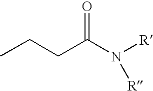
in which R′ and R″ are, independently of one another, hydrogen or C₁-C₆-alkyl, or R′ and R″, together with the nitrogen atom to which they are bonded, are saturated cycloalkyl having 3 to 5 carbon atoms in which a ring member is optionally replaced by oxygen or sulfur and that is optionally substituted by one to four C₁-C₄-alkyl, trifluoromethyl, or C₁-C₄-alkoxy,
comprising contacting a compound of formula (II) 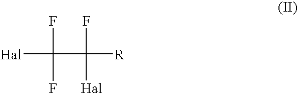
in which
R has the meaning given for formula (I), and
Hal is chlorine, bromine, or iodine, with a dehalogenating agent in a water-comprising polar medium.

9. The method according to claim 8 wherein the polar medium is water.

10. The method according to claim 8 wherein the water-comprising polar medium contains a solubility promoter.

11. The method according to claim 10 wherein the ratio (v/v) of the water-comprising polar medium to the solubility promoter is 1:10 to 1:100.

12. The method according to claim 8 wherein the dehalogenating agent is zinc.

13. The method according to claim 8 wherein the dehalogenating agent is iron.

14. The method according to claim 8 additionally comprising subsequently isolating the compound of formula (I) by distillation from the reaction mixture, and optionally, after phase separation, removing entrained water from the compound of formula (I).
